(19) United States
(12) Patent Application Publication
(10) Pub. No.: US 2025/0367109 A1
(43) Pub. Date: Dec. 4, 2025

(54) DEVICES, SYSTEMS, AND METHODS FOR IMPROVED BONE REMODELING

(71) Applicant: UNIVERSITY OF UTAH RESEARCH FOUNDATION, Salt Lake City, UT (US)

(72) Inventors: Robert Z. Tashjian, Salt Lake City, UT (US); Peter N. Chalmers, Salt Lake City, UT (US); T. Wade Fallin, Hyde Park, UT (US); James M. Hotaling, Salt Lake City, UT (US); Austin M. Hickey, Midvale, UT (US)

(21) Appl. No.: 18/732,532

(22) Filed: Jun. 3, 2024

(51) Int. Cl. A61K 9/00; A61K 31/568; A61K 45/06

(52) U.S. Cl. CPC A61K 9/0024; A61K 31/568; A61K 45/06

(57) ABSTRACT

Disclosed are devices and methods for providing local administration of a sex hormone composition in a mammalian body to promote bone remodeling. The device includes an implantable structure configured for placement at a target intraosseous and/or periosteal site in the mammalian body and an effective amount of a sex hormone composition associated with the implantable structure. The sex hormone composition comprises both an estrogen and an androgen.

### BACKGROUND

Both estrogens (such as estradiol) and androgens (such as testosterone) play important roles in the maintenance of the musculoskeletal system in both men and women. In the prior art, systemic supplementation of an estrogen or androgen has been utilized with an aim to improve the quality of the musculoskeletal system, soft tissue to bone healing, osteoporosis, fracture healing, and implant osseointegration. However, systemic administration exposes anatomical regions distant from the target location to the administered sex hormone and can lead to unwanted side effects.

Accordingly, there is an ongoing need to for new devices, systems, and methods of administering sex hormones to improve bone remodeling, without the often undesirable risks of systemic administration.

### SUMMARY

Disclosed herein are devices, systems, and methods for improving bone remodeling. An effective amount of a composition comprising an estrogen and an androgen may be administered locally at a bone site. The composition provides an increase in the local levels of estrogen and androgen in the subject which leads to improved bone remodeling in proximity to the bone site. The ratio of estrogen to androgen in the composition may be different for male and female subjects, such that the estrogen-to-androgen ratio mimics the physiological estrogen-to-androgen ratio of healthy male and female subjects. The estrogen and androgen used in the composition may be estradiol and testosterone, respectively, and/or their equivalents.

Beneficially, local administration of sex hormones leads to improved bone remodeling in subjects with deficient or normal levels of sex hormones. Subjects that have deficient levels of sex hormones are at higher risk of compromised bone remodeling; thus, the devices, systems, and methods disclosed herein are expected to provide an even greater therapeutic benefit related to bone remodeling for sex hormone deficient subjects than subjects without sex hormone deficiencies.

In some embodiments, the composition is administered by way of a drug-eluting implant implanted at a target bone site. The drug-eluting implant may be implanted in an intraosseous fashion and/or a periosteal fashion. The drug-eluting implant may elute the drug from a coating on the implant, from a reservoir in the implant, or directly from the substrate of the implant. The implant may be a functional implant (e.g., bone plate, suture anchor, intramedullary rod, etc.) or it may be a passive, non-functional implant. In some embodiments, an injection of an extended-release formula comprising the composition may be injected at the target bone site. The composition can thereby elute during at least a portion of a healing period of the bone to sustain improved bone remodeling.

In some embodiments, a drug-eluting implant as disclosed herein is configured for placement at an intraosseous and/or periosteal target site that is not associated with an injured or disordered enthesis. That is, while the embodiments described herein can be utilized for promoting bone remodeling in conjunction with enthesis healing in some circumstances, this is not necessarily the case in all embodiments. Some embodiments may, for example, utilize a device as disclosed herein at a target site not associated with an enthesis, or at least not an injured or disordered enthesis. The embodiments disclosed herein thus encompass applications where enthesis healing is not the intended effect and/or where the subject to be treated does not have an enthesis injury or disorder.

In one embodiment, a device for providing local administration of a sex hormone composition in a mammalian body to promote bone remodeling comprises (i) an implantable structure configured for placement at an intraosseous and/or periosteal target site in the mammalian body, and (ii) an effective amount of a sex hormone composition associated with the implantable structure. The sex hormone composition comprises both an estrogen and an androgen. The device is configured such that the sex hormone composition elutes from the implantable structure under physiological conditions to provide local administration of the sex hormone composition.

The device can be configured with sex-specific ratios of sex hormones to best promote bone remodeling according to the sex of the subject in need of the device. A device configured for a male subject can have a ratio of androgen to estrogen of at least 10:1 and optionally up to 20:1. A device configured for a female subject can have a ratio of androgen to estrogen of at least 1:3 and optionally up to 1:5.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

Various objects, features, characteristics, and advantages of the disclosure will become apparent and more readily appreciated from the following description, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale.

FIG. 1 is a cross sectional view of a soft tissue repaired to a bone, showing an embodiment of a drug-eluting implant.

FIG. 2 is a cross sectional view of an ACL graft fixed in a knee joint, showing an embodiment of a drug-eluting implant.

FIG. 3 is a cross sectional view showing an embodiment of a drug-eluting implant in a bone tunnel in a femur.

FIG. 4 is a cross sectional view showing an embodiment of a drug-eluting implant in a bone tunnel in a humerus.

FIG. 5 is a cross sectional view showing an embodiment of a drug-eluting implant in a bone tunnel in a vertebra.

FIG. 6 is an elevation view showing a bone fixed with a bone plate and bone screws.

FIG. 7 is a cross sectional view showing a joint replacement implant fixed in a femur.

FIG. 8A is an elevation view of an embodiment of a drug-eluting implant in the form of a monolithic implant.

FIG. 8B is an elevation view of an embodiment of a drug-eluting implant in the form of a sheathed implant.

FIG. 8C is an elevation view of an embodiment of a drug-eluting implant in the form of a reservoir implant.

FIG. 8D is an elevation view of an embodiment of a drug-eluting implant in the form of an osmotic pump implant.

FIG. 9 shows an estradiol concentration chart demonstrating that local administration of estradiol led to significantly higher local estradiol concentrations at the target region relative to other regions.

FIG. 10 shows an estradiol release chart comparing estradiol release of two different implant types.

### DETAILED DESCRIPTION

### Introduction

### Impact of Estrogens and Androgens on the Musculoskeletal System

Testosterone is important in both men and women, independent of the indirect effects of testosterone aromatization into estradiol. In male mice orchiectomy alters both number and activity of osteoblasts and osteoclasts. Testosterone and the downstream pathway of the androgen receptor, has an effect on bone quality that is independent of estradiol and aromatization. Androgen-receptor knock out mice have reduced trabecular and cortical bone due to the loss of the suppressive effect of androgens on osteoclastogenesis and osteoclast activation. Androgens also stimulate pre-osteoblasts to differentiate into osteoblasts. Androgen receptors on neuronal cells in bone are also important for maintenance of cortical bone. The effects of androgens on bone are independent of their anabolic effects upon muscle. In human osteoblasts, androgens lead to proliferation and differentiation of osteoblasts. In elderly men, testosterone deficiency is the most important factor in bone loss. In young men testosterone deficiency is the main cause of osteoporosis, and testosterone therapy can improve bone mineral density in these patients. In men, there is a dose-response relationship between testosterone supplementation and bone mineral density. In both men and women, testosterone levels associate with muscle strength. In elderly women, a randomized clinical trial demonstrated that testosterone supplementation improved lean body mass and strength. In elderly men, low testosterone levels are associated with an increased hip fracture risk, a risk which is increased by concomitant low estradiol levels, but not present with low estradiol levels without low testosterone levels. In transgender men, testosterone supplementation improves bone mineral density. These data from cell culture, animal models, and clinical studies are all concordant that testosterone plays an important role in the musculoskeletal system in both men and women.

Estradiol is important in both men and women. In male osteocytes, estrogen receptor deletion is associated with androgen receptor upregulation. In both male and female marrow stromal cells, estradiol increases osteoblast differentiation. Transcriptional data shows that reduced estrogen receptor activity increases osteoblast apoptosis. In small animal models, ovariectomy reduces the number of active osteoblasts and increases the number of active osteoclasts. In men higher estradiol levels are associated with improved bone repair after ceramic cement augmentation of dental defects. Multiple randomized clinical trials have demonstrated that estradiol improves bone mineral density and reduces fracture risk in women. In women, estradiol supplementation has been demonstrated to increase strength, collagen synthesis with exercise, and soft tissue laxity.

However, these effects differ between genders—i.e. the bone cells of men and women respond differently to each hormone. The beneficial effects of both estrogen and testosterone on bone mineral density appear to differ between men and women, as demonstrated by in vitro studies on osteoblasts and osteoclasts. Osteoclasts of men and women display differential gene expression when exposed to estradiol and testosterone. Male and female gonadectomized rats responds differently to estradiol and testosterone treatment, with testosterone having a greater effect on bone in males than females. Women treated with testosterone in addition to estradiol have increased bone mineral density as compared to those treated with testosterone alone. Thus, differential hormonal strategies should be employed for each sex.

There is evidence that balance between hormones is also a factor. Androgen-receptor over-expressing mice have reduced osteoblast activity. Higher than normal testosterone levels also lead to an increased risk for fracture clinically. In a clinical study in elderly men, combined treatment with both hormones had the greatest effect upon circulating bone-turnover factors. In post-menopausal women, alteration of the estradiol to testosterone ratio increased the risk for osteoporosis. In our own previous study, estradiol and testosterone had differential beneficial gene expression and clinical effects upon rotator cuff repair. (Tashjian et al. Estrogen and testosterone supplementation improves tendon healing and functional recovery after rotator cuff repair. J Orthop Res. Published online 2023.) Thus, achieving the right balance between hormones will achieve the optimal benefits to the musculoskeletal system.

### Impact of Estrogens and Androgens on Osteoporosis

Osteoporosis is one of the most common causes of fracture and disability in elderly individuals. Osteoporosis is a dysregulation of the homeostasis of bone turnover—i.e. a loss of the balance between osteoblasts and osteoclasts. It is estimated that one third of post- menopausal women will suffer a fragility fracture. These fractures are morbid, as within two years of an osteoporosis-related proximal humerus fracture 24% of patients will be dead and within one year of an osteoporosis-related hip fracture, 25 to 36% of patients will be dead. While systemic estradiol can prevent these types of fractures, it leads to an overall increase in cardiovascular morbidity. The current dominant treatment strategy, bisphosphonates, only reduce osteoclast activity and thus lead to side effects such as stress fractures. There is thus an urgent need for new, innovative preventative treatments for osteoporosis.

There is extensive evidence demonstrating that both estrogen and testosterone deficiency cause osteoporosis. Multiple animal studies have shown that ovariectomy and orchiectomy result in osteoporosis. Multiple human studies have demonstrated a correlation between the post-menopausal status in women and testosterone deficiency in men and osteoporosis. Multiple human studies have also demonstrated that estrogen treatment and testosterone treatment improve bone mineral density.

### Impact of Estrogens and Androgens on Fracture Healing

Epidemiologic studies of hip and spine fractures report significant differences between men and women with respect to fracture incidence and healing and sex hormone deficiency. Similarly, clinical studies report that males show more rapid fracture healing and have a high incidence of hypertrophic non-unions compared to atrophic non-unions which are more commonly observed in females. Animal data supports that bone fracture healing is different between adult male and female mice with a stronger healing response in male compared to female mice. Long-term estrogen deficiency has been found to impair fracture healing with reduced force required to break a healed osteotomy and poorer histology compared to non-estrogen deficient animals in a rat model. Testosterone deficiency induced by excessive opioids has also been shown to impair fracture healing in a rat model. Androgen receptors are highly expressed in bone periosteum and elimination of androgen receptors in the periosteum in a mouse fracture model significantly impaired fracture healing.

Estrogen and testosterone act differently in the setting of fracture healing. Fracture healing occurs in stages where initially a hematoma is formed, inflammatory cells are recruited, mesenchymal stem cells migrate into the fracture region, cells differentiate into cartilaginous tissue, further maturation of the soft callus occurs with vascularization and mineralization yielding trabecular bone which is then matured by osteoblasts and osteoclasts into lamellar bone. Estrogen acts directly on stem cells promoting differentiation. Estrogen also inhibits osteoclast maturation through upregulation of osteoprotegerin (OPG) binding to RANK-L. Estrogen also inhibits osteoclast activity by upregulation of VEGF. Estrogen simultaneously prevents osteoblast apoptosis. Testosterone also inhibits osteoclast maturation through OPG and upregulates osteoblast differentiation. In the setting of fracture, estrogen and testosterone play key roles in the processes of fracture healing. Estrogen deficiency is associated with prolonged inflammation and recruitment of neutrophils not allowing later repair and remodeling of the callus. It is also associated with the production of IL-12 and IL-18, enhancing TNF alpha production increasing osteoclast activity resulting in bone loss. Therefore, estrogen deficiency not only prolongs the inflammatory phases of fracture healing but also impairs remodeling through a reduction in angiogenesis and revascularization of the soft callus. Testosterone directly stimulates osteoblasts resulting in increased mineralization as well as inducing IGF-1 expression which plays a crucial role in fracture healing as well through reducing osteoblast apoptosis and promoting osteoblastogenesis by stabilizing beta-catenin, enhancing WNT-dependent activity.

### Impact of Systemic and Local Estrogens and Androgens Supplementation on Improving Fracture Healing

Bones demonstrate a remarkable ability to regenerate following fracture injury. recovering from structural failure and lost physiological function. Despite this innate ability for fractures to heal, the process may be impaired. Currently, 10-15% of the approximately 15million fractures that occur annually result in poor or unresolved healing. As the aging population is expected to double by 2050 and the occurrence of osteoporotic fractures rise in the near future, impairment in osteoporotic fracture healing is becoming an emerging public health concern. Moreover, it has previously been reported that the risk of non-union increases with age and that osteoporotic fracture is associated high morbidity. mortality rate and increased healthcare costs. Few biologic options exist to improve healing in the setting of osteoporotic bone, non-united fractures or fractures at-risk anatomically for healing failure. Bone morphogenic protein-2 and -7 are the only available local biologic options to improve fracture healing and have limited on-label applications, and off-label usage, while common. has limited evidence to support its efficacy.

Supplementation of estrogen and testosterone both systemically as well locally has been determined to improve fracture healing in animal models. Systemic supplementation of testosterone has been shown to improve calcium callus concentrations in a rat fracture model compared to controls supporting the beneficial effects of systemic testosterone on healing. Testosterone delivered locally at the site of fracture using a polypropylene fumarate/tricalcium phosphate scaffold was determined to be as effective as bone morphogenic protein-2 in improving callus formation and bone volume in comparison to controls in a mouse fracture model. In a rabbit fracture model, locally injected estrogen at the site of a stabilized fracture was found to lead to improved fracture stability, radiographic union rates and gap reduction compared to controls as well as systemic estrogen supplementation. Estradiol-loaded Poly(e-caprolactone)/silk fibroin electrospun microfibers were noted to reduce osteoclast activity in cell culture. Finally, addition of an estradiol eluting PLGA nanoparticle scaffold locally at the site of a fracture in an ovarectomized mouse has been found to not increase systemic levels of estrogen but has been shown to improve overall bone volume and strength of repaired fracture in comparison to ovarectomized animal controls. The addition of the local estrogen was determined to improve the strength of the repair back to the level of strength determined in non-ovarectomized animals.

### Impact of Systemic and Local Estrogen and Testosterone Supplementation on Implant Osseointegration

Failure of osseointegration remains a major challenge within orthopaedic surgery. Aseptic loosening remains a common cause of failure with modern upper and lower extremity arthroplasty implants. In the only randomized study compared cemented and uncemented shoulder arthroplasty, cemented fixation was superior. Thus, cement fixation remains common, despite many decades of innovation surrounding cementless, osseointegrated components.

The literature surrounding implant osseointregration and sex hormone deficiency and hormone replacement therapy is extensive. Multiple studies have demonstrated that estrogen deficiency reduces osseointegration of titanium implants in cancellous bone in rats, mice, dogs, and sheep. A similar effect has been observed with testosterone. In addition, multiple studies have demonstrated that systemic administration of estradiol improves osseointegration in cancellous bone in estrogen-deficient rats. A similar effect is observed with selective-estrogen-receptor modulators. This affect appears to be greater than observed with calcitonin or alendronate although in some studies, alendronate was as or more effective or synergistic. This effect appears to have been verified clinically, as in women examined in a registry study, hormone replacement therapy was associated with a 40% reduced risk for early revision after hip and knee arthroplasty as compared to matched controls. Similarly, the risk for dental implant failure doubles without hormone supplementation in post-menopausal females. Thus, there is extensive evidence to support hormone replacement therapy to improve osscointegration.

### Localized Administration of Both an Estrogen & an Androgen

Overall, the prior art supports the rationale for systemic supplementation of a sex hormone (an estrogen or an androgen) to improve the quality of the musculoskeletal system, soft tissue to bone healing, osteoporosis, fracture healing, and implant osscointegration (collectively “bone remodeling”). However, what has not been suggested in the prior art is the need for local supplementation of specific combinations of sex hormones, specifically a combination of an estrogen and an androgen, to address bone remodeling. Accordingly, there is a need to provide new devices, systems, and methods that provide novel combinations of sex hormones to further improve and optimize bone remodeling.

### Sex Hormones & Physiological Ratios

The devices, systems, and methods disclosed herein are configured to locally administer an estrogen and an androgen to promote bone remodeling. Endogenous estrogens include estrone, estradiol, and estriol. Endogenous androgens include testosterone, dihydrotestosterone, and androstenedione. Exogenous estrogens include synthetic alternatives, derivatives, estrogen esters, and estrogen ethers. Exogenous androgens include synthetic alternatives, derivatives, androgen esters, and androgen ethers. As used herein, an estrogen or androgen “equivalent” includes synthetic or synthetically modified sex hormones, as well as compounds that function to promote levels of sex steroids within the body via receptor modulation (e.g., selective sex steroid receptor modulators), inhibiting breakdown or conversion of sex steroids, and the like. Selective estrogen receptor modulators include afimoxifene, arzoxifene, bazedoxifene, clomifene, femarelle, lasofoxifene, ormeloxifene, tamoxifen, toremifene, analogs, and derivatives thereof. Selective androgen receptor modulators include enobosarm, ligandrol, testolone, andarine, S23, analogs, and derivatives thereof. The terms “estrogen” and “androgen” will be understood to broadly encompass their endogenous and exogenous forms and their equivalents unless specified otherwise. Any combination of the foregoing may be utilized. Where specific examples of dosages or ratios are provided in the context of testosterone and estradiol, the same dosages and ratios can be applied to other embodiments where other estrogen(s) and/or androgen(s) are utilized.

Testosterone (T) levels in men peak shortly after puberty and decline throughout the lifespan at roughly 10% a decade with marked decreases each decade, particularly after 40 years old. Similarly, bone health also peaks in younger men and declines throughout the lifespan. Hypogonadism, defined as low testosterone, is one of the drivers of bone health in men. Estrogen and the ratio of testosterone to estradiol (E2) are also crucial to overall bone health, as well as sexual health. Mean morning total testosterone levels in 25-30 year old men are 519 ng/dl. Typical estradiol levels are 25 pg/ml in young healthy males. The urologic literature typically quotes a ratio of T:E2 of >10:1 being optimal for reproduction. In young healthy males, we hypothesize that an optimal ratio for bone health is T:E2 at a ratio of 10:1 to 20:1 or in some instances greater than 20:1 (higher ratios in the male context mean higher androgen relative to estrogen). Accordingly, embodiments disclosed herein and intended for use in a male subject may be configured to include a T:E2 ratio of 5:1 to 30:1, or 7.5:1 to 25:1, or 10:1 to 20:1, such as 12.5:1, 15:1, or 17.5:1, or a ratio within a range defined by any two of the foregoing as endpoints.

Female reproductive hormones vary throughout the lifespan and with the menstrual cycle. Estradiol is usually 30-40 pg/mL at the beginning of the cycle (cycle day ˜1-3). It peaks at 200-500 pg/mL just prior to LH surge. The average is 100 pg/mL. Testosterone in young women is typically 30.5. Given that female bone health also tends to peak early in adulthood and declines throughout the lifespan we hypothesize that an optimal ratio of T:E2 is at least 1:3 and optionally up to 1:5 (higher ratios in the female context mean higher estrogen relative to androgen). Accordingly, embodiments disclosed herein and intended for use in a female subject may be configured to include a T:E2 ratio of 1:2 to 1:8, or 1:2.5 to 1:6, or 1:3 to 1:5, such as 1:3.5, 1:4, or a ratio within a range defined by any two of the foregoing as endpoints.

### Drug-Eluting Implants & Coatings

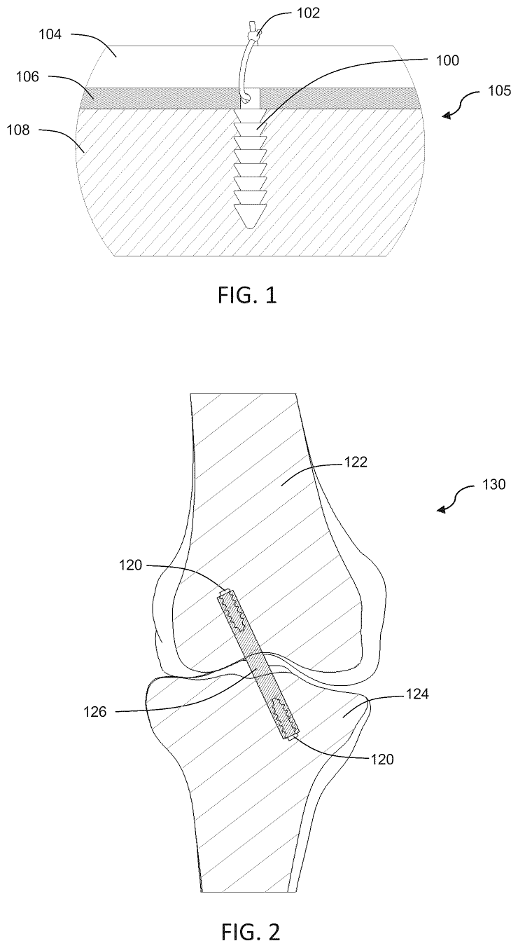

Referring to FIG. 1, a drug-eluting implant 100 may be provided in the form of a suture anchor. The suture anchor may be in the form of a barbed cylindrical device as shown, or may be in the form of a threaded device, toggle device, press fit device, expanding suture device, or any other form known in the art. Suture 102 may be attached to drug-eluting implant 100 and is used to attached soft tissue 104 to a surface of a bone 105 which may be comprised of cortical bone 106 and cancellous bone 108. Soft tissue 104 may be comprised of a tendon or a ligament.

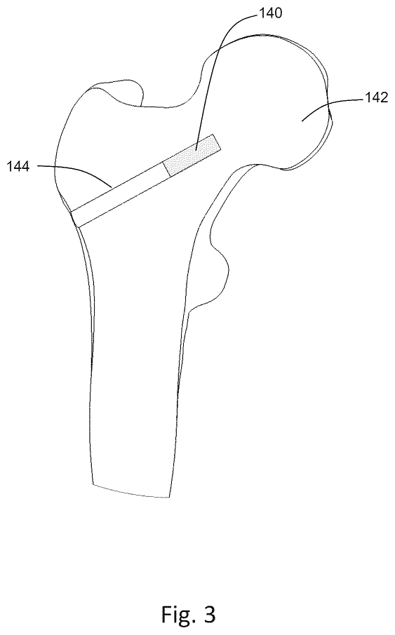

Referring to FIG. 2, a drug-eluting implant 200 may be provided in the form of a fixation device for an ACL graft 126 in a knee joint 130 where a femur 122 articulates with a tibia 124. “ACL” refers to an anterior cruciate ligament. The ACL graft 126 may be located in bone tunnels located in femur 122 and tibia 124, and drug-eluting implants 200 may be positioned in or proximate to the bone tunnels to fix the ACL graft 126 to the femur 122 and tibia 124, respectively, in an intraosseous fashion. The fixation device for an ACL graft 126 is shown in the form of an interference screw, or may be in the form of a suture and button construct, or any other form known in the art.

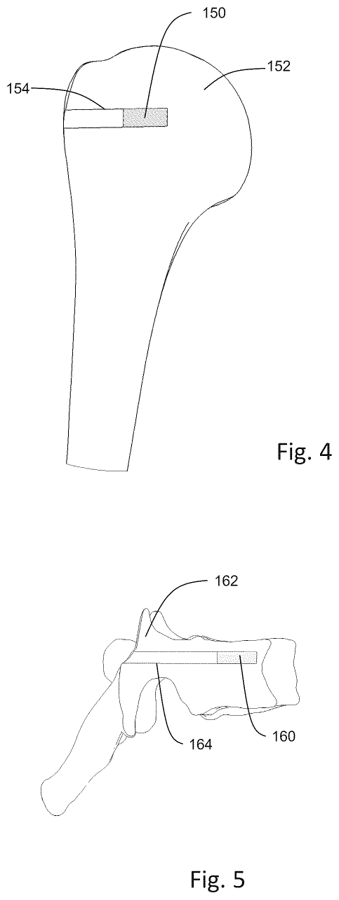

FIG. 3 shows a drug-eluting implant 140 positioned in a bone tunnel 144 in femur 142. FIG. 4 shows a drug-eluting implant 150 positioned in a bone tunnel 154 in humerus 152. FIG. 5 shows a drug-eluting implant 160 positioned in a bone tunnel 164 in vertebra 162.

Drug-eluting implants 100, 120, 140, 150, and 160 may be comprised of a biomaterial and one or more sex hormones (also known as sex steroids, gonadal steroids, and gonadocorticoids). Biomaterials may be comprised of a biocompatible material that is biological, metal, ceramic, carbon, a single polymer, a copolymer, a polymer blend, or combinations thereof. Carbon materials include carbon fiber, pyrolytic carbon, and others known in the art. Biological materials may be selected from bone, collagen, ligaments, cartilage, dermal tissue, amniotic tissue, and others known in the art. Biocompatible metals may be selected from titanium and alloys thereof, magnesium and alloys therof, cobalt alloys, stainless steels, and others known in the art. Biocompatible ceramics may be selected from bioactive glass, hydroxyapatite, tricalcium phosphate, alumina, zirconia, and others known in the art. Biocompatible polymers may be selected from polyetheretherketone, polyetherketoneketone, polyethylene, polyester, polypropylene, silk, nylon, polyglyconate, polydioxanone, polyglactin, polyglycolic acid, polylactic acid, polylactide-co-glycolide, polydioxanone, polydroxyalkanoate, poliglecaprone, polycaprolactone, or others known in the art. Sex hormones may be selected from androgens, estrogens, and progestogens.

In one embodiment, drug-eluting implants 100, 120, 140, 150 and 160 may be made from a biodegradable polymer mixed with a sex hormone, or combinations of sex hormones. The biodegradable polymer may be selected from polyglycolic acid, polylactic acid, and polycaprolactone, and combinations thereof. The sex hormone may be selected from testosterone or its equivalents, and estrogen, or its equivalents, and combinations thereof. For use in a male subject, the sex hormones may be provided in a mass ratio in a range from 10:1 to 20:1 of testosterone to estradiol. For use in a female subject, the sex hormones may be provided in a mass ratio in a range from 5:1 to 3:1 of estradiol to testosterone. The drug-eluting implants 100, 120, 140, 150, and 160 may be fabricated by a compounding process followed by a molding process. The compounding process for forming a drug-loaded polymer may include the steps of solvent mixing the sex hormone(s) with the polymer, followed by removal of the solvent from the mixture by evaporation, heat, vacuum, combinations thereof, or other means known in the art. An alternate compounding process may be to use a pharmaceutical twin-screw extruding machine to mix the polymer and sex hormone(s). The molding process may include the steps of pelletizing the drug-loaded polymer, followed by an injection molding, compression molding, or other molding processes known in the art, to convert the pelletized drug-loaded implant into a desired shape. The final drug-loaded implant is configured to elute the drug, in this case the sex hormone(s), over a desired period after implantation into a mammalian body. The elution of the sex hormone(s) will enhance bone remodeling locally in the tissues in proximity to the drug-eluting implants, as discussed in the background section above.

In an alternate embodiment, drug-eluting implants 100, 120, 140, 150, and 160 may be made using the esterified sex hormone(s) to create an injectable prodrug that will elute the sex hormone(s) over a desired period of time. In this embodiment, the drug-eluting implant may be injected using standard syringe injection techniques. Sex hormones may be provided in the ratios described above.

In an alternate embodiment, drug-eluting implants 100, 120, 140, 150, and 160 may be made from a biocompatible thermo-sensitive hydrogel mixed with a sex hormone(s), where the thermo-sensitive hydrogel is in liquid form at room temperature, and a gel form at body temperature, and where the sex hormone will be eluted over a desired period of time. In this embodiment, the drug-eluting implant may be injected using standard syringe injection techniques. Sex hormones may be provided in the ratios described above.

As shown in FIG. 1, following surgery to reattach soft tissue 104 that has torn from its attachment site to bone 105 using drug-eluting implant 100 and suture 102, drug-eluting implant 100 may be used to provide local delivery of a sex hormone(s) as a therapeutic agent to enhance local bone remodeling as soft tissue 104 heals to the bone 105. Following surgery, drug-eluting implant 100 may release the sex hormone(s) for a period of time, generally between one and twelve months, and preferably between two to three months, to enhance local bone remodeling. Therapeutically enhanced bone remodeling will provide increased strength of the healed interface between soft tissue 104 and bone 105 and reduce the risk of recurrence or retear and increase the durability of the repair.

As shown in FIG. 2, following surgery to attach both ends of ACL graft 126 to the femur 122 and tibia 124 using drug-eluting implant 120, drug-eluting implant 120 may be used to therapeutically enhance local bone remodeling as the ACL graft 126 heals to the femur 122 and tibia 124. Following surgery, drug-eluting implant 120 may release the sex hormone(s) for a period of time, generally between one and twelve months, and preferably between two to three months, to therapeutically enhance local bone remodeling. Enhanced bone remodeling due to the eluted drug will provide increased strength of the healed interface between the ACL graft 126, the femur 122, and tibia 124 to reduce the risk of recurrence or retear and to increase the durability of the repair. Drug-eluting implant 120 may be used for any intra-osseous fixation of a ligament or a tendon to a bone.

As shown in FIGS. 3, 4, and 5, drug-eluting implants 140, 150, and 160 may be used to locally treat osteoporosis, providing local prophylactic therapeutic treatment to prevent bone fracture. Drug-eluting implant 140 may be placed into a bone tunnel 144 in a femur 142 as a local therapeutic prophylactic treatment to prevent proximal femur fractures that are common in people with osteoporosis or metabolic bone disorders. Drug-eluting implant 150 may be placed into a bone tunnel 154 in a humerus 152 as a local therapeutic prophylactic treatment to prevent proximal humeral fractures that are common in people with osteoporosis or metabolic bone disorders. Drug-eluting implant 160 may be placed into a bone tunnel 164 in a vertebra 162 as a local therapeutic prophylactic treatment to prevent vertebral compression fractures that are common in people with osteoporosis or metabolic bone disorders. Desired elution periods for drug-eluting implants 140, 150, and 160 may range from months to years. Local prophylactic therapeutic treatment to prevent proximal bone fractures may be considered anywhere in the body where osteoporosis or metabolic bone disease exists.

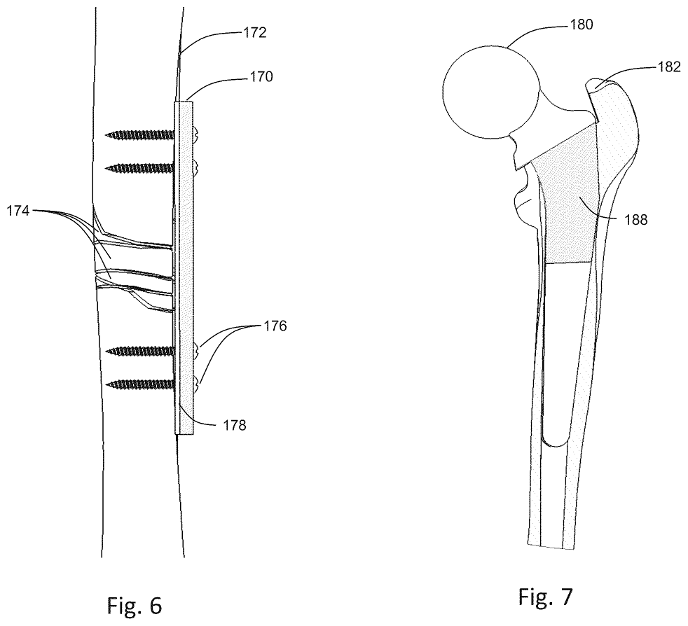

Referring to FIG. 6, a bone 107 may have a zone of fracture occupied by bone fragments 174. A bone plate 170 and bone screws 176 are used to fix and stabilize bone 172 and bone fragments 174 in a correctly aligned position. Bone plate 170 may have a drug-eluting coating 178 comprised of a polymer, as described above, and a sex hormone(s). Following a bone plating surgery, drug-eluting coating 178 of bone plate 170 may release the sex hormone(s) for a period of time, generally between one and twelve months, and preferably between two to three months, to enhance local bone remodeling. Enhanced therapeutic bone remodeling due to the eluted drug provides more rapid bone repair and increased strength of the healed zone of fracture.

In FIG. 7, a joint replacement implant 180 in the form of a femoral component of a total hip replacement is shown implanted into a femur 182. Joint replacement implant 180 may have a drug-eluting coating 188 comprised of a polymer, as described above, and a sex hormone(s). Drug-eluting coating 188 may be applied directly to a surface of joint replacement implant 180, or it may be applied to a porous coating (not shown) that is attached to the surface of joint replacement implant 180. Following a joint replacement surgery, drug-eluting coating 188 of joint replacement implant 180 may release the sex hormone(s) for a period of time, generally between one and twelve months, and preferably between two to three months, to enhance local bone remodeling. Enhanced bone remodeling due to the eluted drug provides improved osteointegration of joint replacement implant 180 to the femur 182, and similarly, improved osteointegration can be achieved for joint replacement implants used in any joint in the body.

Drug-eluting coatings 178 and 188 may be fabricated by a variety of methods, including dip coating, spray coating, electrostatic coating, vapor deposition, molding, and other methods known in the art.

In one embodiment, drug-eluting coatings 178 and 188 may be made from a biodegradable polymer mixed with a sex hormone, or combinations of sex hormones. The biodegradable polymer may be selected from polyglycolic acid, polylactic acid, and polycaprolactone, and combinations thereof. The sex hormone may be selected from testosterone and estrogen, and combinations thereof. For use in a male subject, the sex hormones may be provided in a mass ratio in a range from 10:1 to 20:1 of testosterone to estradiol. For use in a female subject, the sex hormones may be provided in a mass ratio in a range from 5:1 to 3:1 of estradiol to testosterone.

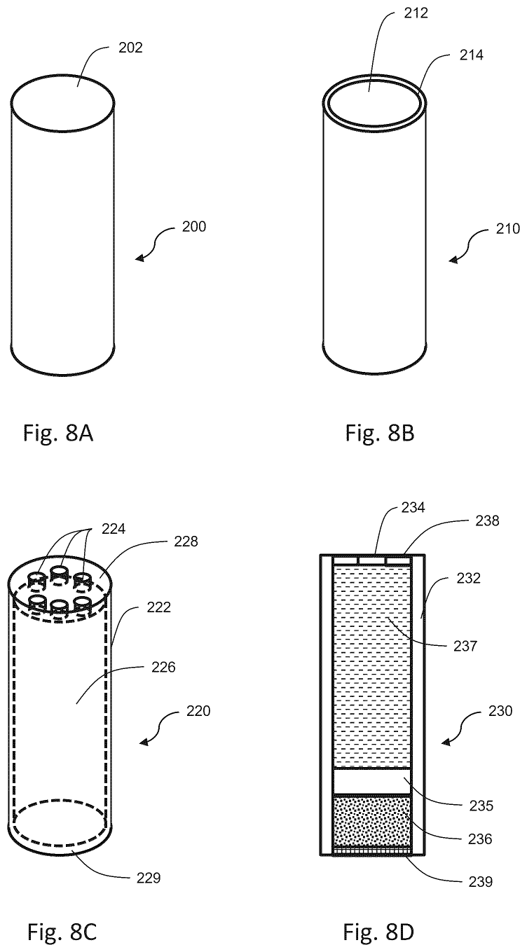

Drug-eluting implants 100, 120, 140, and 160 may be provided in a variety of configurations. FIG. 8A shows a drug-eluting implant 200 shown in FIG. 8A, having a body 202. Body 202 is in the shape of a cylinder, but any desirable shape may be used. Body 202 may be comprised of a polymer and a sex hormone(s). Drug-eluting implant 200 is also referred to as a monolithic implant. FIG. 8B shows a drug-eluting implant 210 having a sheath 212 and a core 214. Sheath 212 may consist of a polymer, and core 214 may be comprised of the same or different polymer and a sex hormone(s). Drug-eluting implant 210 is also referred to as a sheathed implant. FIG. 8C shows a drug-eluting implant 210 having an internal cavity 226 bounded by an outer wall 222, first end cap 228, and second end cap 229, all comprised of a biomaterial as previously described. Internal cavity 226 may be filled with a sex hormone(s). Ports 224 may provide for a controlled elution rate of the sex hormone(s) from internal cavity 226. Ports 224 are shown extending through first end cap 228, but they may be located on outer wall 222, second end cap 229, or any combination thereof. Ports 224 may be provided as a single port or multiple ports, in one or more sizes, to provide for a desired elution profile of the sex hormone(s) over time. Drug-eluting implant 220 is also referred to as a reservoir implant.

As shown in FIG. 8D, drug-eluting implant 230 may be comprised of a first end cap 238, second end cap 239, and outer wall 232 that form an outer shell. The outer shell contains an internal cavity 237 which may be filled with a sex hormone(s), a piston 235 and an osmotic layer 236. First endcap 238 may have a single port 234 or multiple ports (not shown). Alternatively, first end cap 238 may be compromised of a porous material or a semi-permeable membrane (not shown) to for a desired elution profile of the sex hormone(s) over time. Second end cap 239 may be comprised of a porous material or a semi-permeable membrane to allow for fluid ingress into osmotic layer 236 to allow for expansion of osmotic layer 236 to urge piston 235 towards first end cap 238 to elute the sex hormone(s) from drug-eluting implant 230. Drug-eluting implant 230 is also referred to as an osmotic pump implant.

### Working Examples

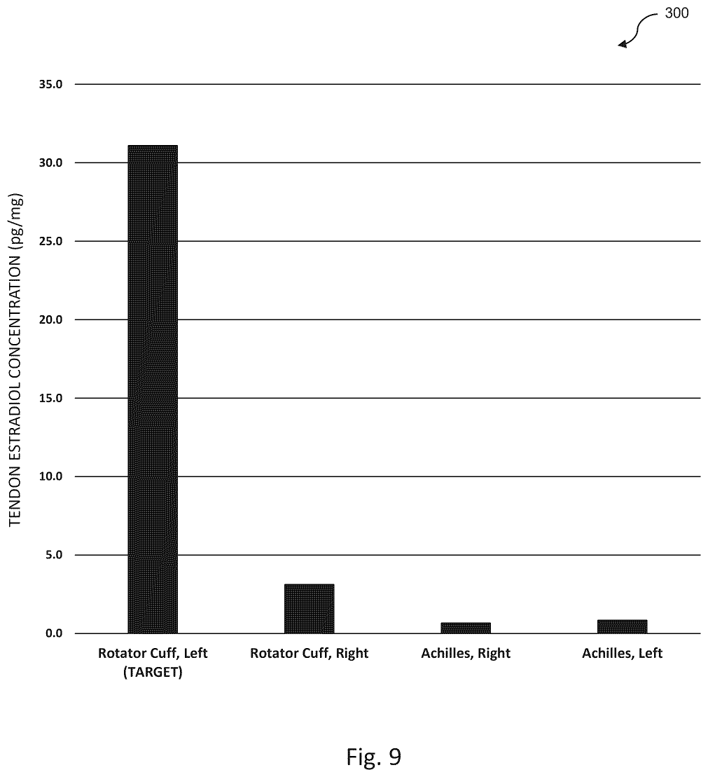

With reference to FIG. 9, in a first experiment, local elution of a sex hormone, estradiol, was studied. Drug-eluting implants analogous to drug-eluting implant 200 were comprised of polycaprolactone and estradiol. The drug-eluting implants were in the form of a cylinder 1 mm in diameter and 5 mm long, and loaded with 200 micrograms of estradiol. The drug-eluting implants were implanted into the left proximal humeri of wild-type rats. One week following implantation, the animals were euthanized and tendon tissue samples were collected at the following sites: left rotator cuff (target site), right rotator cuff, left Achilles tendon, and right Achilles tendon. Mass spectroscopy was used to determine the estradiol concentration in the tissue samples, and the results are provided in an estradiol concentration chart 300. As shown in estradiol concentration chart 300, the left rotator cuff tissue sample, immediately adjacent to the left proximal humerus where the drug-eluting implant was implanted, demonstrated an order of magnitude higher concentration of estradiol, thus demonstrating an effective local administration of the sex hormone. While this experiment only used estradiol, it is expected that any sex hormone or combination of sex hormones loaded into the drug-eluting implant would exhibit a similar effect of local administration. Accordingly, levels of the estrogen and the androgen may be significantly higher (e.g., at least 2, 5, 10, 15, 20, or 25 times higher, or higher by a factor within a range of any combination of the foregoing values) at the anatomical region associated with the target site than at anatomical regions remote from the target site during the treatment duration.

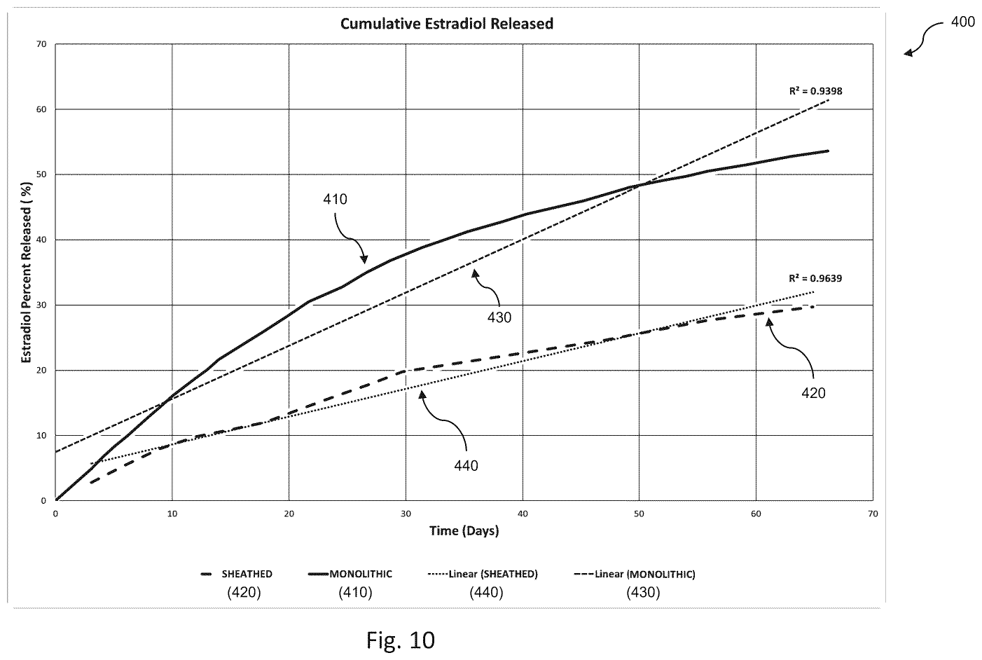

With reference to FIG. 10, in a second experiment, the elution profile of different drug-eluting implant configurations was studied. A first drug-eluting implant design was in the form of drug-eluting implant 200 (monolithic implant), and a second drug-eluting implant design was in the form of drug-eluting implant 210 (sheathed implant). The drug-eluting implants were placed in simulated body fluid (buffered phosphate solution) at body temperature (37 degrees Celsius) and periodic samples were taken to determine the percent of drug release. Both implants were in the shape of a cylinder 4 mm in diameter and 10 mm long. The monolithic implant was comprised of polycaprolactone and 6.8 milligrams of estradiol, and the sheathed implant had a sheath comprised of polycaprolactone and a core comprised of polycaprolactone and 7.8 milligrams of estradiol. The percent of total mass of estradiol released is shown in curve 410 for the monolithic implant and in curve 420 for the sheathed implant. A linear least squared first regression line 430 having a correlation coefficient of 0.9398 is shown for curve 410, and a second regression line 440 having a correlation coefficient of 0.9639 is shown for curve 420. Thus, for bone remodeling processes that would benefit from higher doses of sex hormone local elution during the early healing period, a monolithic implant may be chosen. For bone remodeling processes that would benefit from consistent doses of sex-hormone local elution during the entire healing period, a sheathed implant may be chosen.

### Additional Terms & Definitions

While certain exemplary embodiments of the present disclosure have been described in detail, with reference to specific configurations, parameters, components, elements, etcetera, the descriptions are illustrative and are not to be construed as limiting the scope of the claimed invention. The word “exemplary” is used herein to mean “serving as an example, instance, or illustration.” Any embodiment described herein as “exemplary” is not necessarily to be construed as preferred or advantageous over other embodiments.

For purposes of interpreting this specification, the following definitions will apply. In the event that any definition set forth below conflicts with any document incorporated herein by reference, the definition set forth below shall control.

An “effective dose” or “effective amount” is a dose that provides measurable therapeutic benefits when administered to a subject using one of the administration methods described herein. In particular, an effective dose of sex hormones is a dose that provides a measurable improvement to bone remodeling as compared to bone not treated with the sex hormones. Specific, exemplary dosages are provided herein; however, an effective dose may lie outside the specifically described examples depending on specifics such as subject anatomy, procedure needs, extent of condition, and the like.

As used herein, “physiological conditions” are the conditions expected at and near a target intraosseous or periosteal site in a mammalian body, and typically includes a pH of 6 to 8 (e.g., typically about 7.4 within bone tissue) and with physiological ion concentrations and osmolarity (e.g., about 285 to 295 mOsm/kg).

As used herein, a “target site” is an anatomical location in which a device as disclosed herein is intended for placement, including at least the immediately adjacent bone tissues where bone remodeling occurs following placement of a disclosed device.

Intraosseous means internal to a bone structure. Periosteal means in proximity to a bone surface. Therapeutically enhanced bone remodeling means that bone mineral density will increase and/or bone mechanical strength will increase in response to the therapeutic agent when compared to bone not treated by the therapeutic agent. Therapeutic agents may include the sex hormones disclosed herein at the ratios and/or doses disclosed herein. Soft tissue refers to ligaments and tendons.

Furthermore, for any given element of component of a described embodiment, any of the possible alternatives listed for that element or component may generally be used individually or in combination with one another, unless implicitly or explicitly stated otherwise.

The various features of a given embodiment can be combined with and/or incorporated into other embodiments disclosed herein. Thus, disclosure of certain features relative to a specific embodiment of the present disclosure should not be construed as limiting application or inclusion of said features to the specific embodiment. Rather, it will be appreciated that other embodiments can also include such features.

In addition, unless otherwise indicated, numbers expressing quantities, constituents, distances, or other measurements used in the specification and claims are to be understood as optionally being modified by the term “about.” When the terms “about,” “approximately,” “substantially,” or the like are used in conjunction with a stated amount, value, or condition, it may be taken to mean an amount, value or condition that deviates by less than 20%, less than 10%, less than 5%, less than 1%, less than 0.1%, or less than 0.01% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

The embodiments disclosed herein should be understood as comprising/including disclosed components, and may therefore include additional components not specifically described. Optionally, the embodiments disclosed herein are essentially free or completely free of components that are not specifically described. That is, non-disclosed components may optionally be completely omitted or essentially omitted from the disclosed embodiments. For example, therapeutic agents that are not specifically disclosed herein may independently be optionally omitted. Examples of therapeutic agents that can independently optionally be included or independently optionally omitted include other hormones (i.e., non-sex hormones) such as calcitonin and parathyroid hormone and their analogues, osteoclast modifiers such as bisphosphonates, peptides, growth factors, and antibodies (e.g., anti-sclerostin antibodies, anti-RANKL antibodies). Sex hormone equivalents such as selective estrogen receptor modulators (SERMs) and/or selective androgen receptor modulators (SARMs) may also be optionally omitted.

An embodiment that “essentially omits” or is “essentially free of” a component may include trace amounts and/or non-functional amounts of the component. For example, an “essentially omitted” component may be included in an amount no more than 1%, no more than 0.1%, or no more than 0.01% by total weight of the implant device.

A composition that “completely omits” or is “completely free of” a component does not include a detectable amount of the component (i.e., does not include an amount above any inherent background signal associated with the testing instrument) when analyzed using standard analysis techniques such as, for example, chromatographic techniques (e.g., thin-layer chromatography (TLC), gas chromatography (GC), liquid chromatography (LC)), molecular techniques (e.g., Western blotting, ELISA, immunohistochemistry, flow cytometry, immunoprecipitation), or spectroscopy techniques (e.g., Fourier transform infrared (FTIR) spectroscopy).

1. A device for providing local administration of a sex hormone composition in a mammalian body to promote bone remodeling, the device comprising:
an implantable structure configured for placement at an intraosseous and/or periosteal target site in the mammalian body; and
an effective amount of a sex hormone composition associated with the implantable structure, the sex hormone composition comprising both an estrogen and an androgen, both of which are included in the implantable structure,
wherein the device omits other growth factors, and
wherein the device is configured such that the sex hormone composition elutes from the implantable structure under physiological conditions to provide local administration of the sex hormone composition.

2. The device of claim 1, wherein the device is configured to elute the sex hormone composition for a period of one month to twelve months under physiological conditions.

3. The device of claim 1, wherein the device is configured to elute the sex hormone composition for a period of two months to three months under physiological conditions.

4. The device of claim 1, wherein the device is configured for use in a male subject and wherein a ratio of androgen to estrogen in the sex hormone composition is at least 10:1 and optionally up to 20:1.

5. The device of claim 1, wherein the device is configured for use in a female subject and wherein a ratio of androgen to estrogen in the sex hormone composition is at least 1:3 and optionally up to 1:5.

6. The device of claim 1, wherein:
the estrogen comprises one or more of an endogenous estrogen, an exogenous estrogen, or an estrogen equivalent; and/or
wherein the androgen comprises one or more of an endogenous androgen, an exogenous androgen, or an androgen equivalent.

7. The device of claim 6, wherein:
the estrogen equivalent includes one or more of afimoxifene, arzoxifene, bazedoxifene, clomifene, femarelle, lasofoxifene, ormeloxifene, tamoxifen, toremifene, or analogs and derivatives thereof; and/or
the androgen equivalent includes one or more of enobosarm, ligandrol, testolone, andarine, S23, or analogs and derivatives thereof.

8. The device of claim 1, wherein the estrogen comprises estradiol.

9. The device of claim 1, wherein the androgen comprises testosterone.

10. The device of claim 1, wherein the device is configured to, when positioned at the target site, promote one or more of soft tissue to bone healing, fracture healing, or osteointegration of a bone implant device.

11. The device of claim 1, wherein the device is configured to, when positioned at the target site, reduce a degree of osteoporosis.

12. The device of claim 1, wherein the implantable structure is configured as a monolithic implant, a sheathed implant, a reservoir implant, or an osmotic pump implant.

13. The device of claim 1, wherein at least a portion of the implantable structure is formed from a polymer substrate and wherein at least a portion of the sex hormone composition is embedded within the polymer substrate.

14. The device of claim 1, wherein the implantable structure further comprises a coating and wherein at least a portion of the sex hormone composition is disposed in the coating.

15. The device of claim 1, wherein the implantable structure further comprises a gel and wherein at least a portion of the sex hormone composition is disposed in the gel.

16. The device of claim 1, wherein the implantable structure further comprises a prodrug and wherein at least a portion of the sex hormone composition is disposed in the prodrug.

17. A device for providing local administration of a sex hormone composition in a mammalian body to promote bone remodeling, the device comprising:
an implantable structure configured for placement at an intraosseous and/or periosteal target site in the mammalian body; and
an effective amount of a sex hormone composition associated with the implantable structure, the sex hormone composition comprising both an estrogen and an androgen,
wherein the device is configured such that the sex hormone composition elutes from the implantable structure under physiological conditions to provide local administration of the sex hormone composition, and
wherein the device is specifically configured for a male subject or a female subject,
(i) wherein when the device is configured for a male subject, the sex hormone composition comprises a ratio of androgen to estrogen of at least 10:1 and optionally up to 20:1,
(ii) wherein when the device is configured for a female subject, the sex hormone composition comprises a ratio of androgen to estrogen of at least 1:3 and optionally up to 1:5.

18. The device of claim 17, wherein the device is configured to elute the sex hormone composition for a period of one month to twelve months under physiological conditions.

19. The device of claim 17, wherein the estrogen comprises estradiol and wherein the androgen comprises testosterone.

20. The device of claim 17, wherein the implantable structure is configured as a monolithic implant, a sheathed implant, a reservoir implant, or an osmotic pump implant.

21. A method of providing local administration of a sex hormone composition in a mammalian body to promote bone remodeling, the method comprising:
identifying a mammalian subject as male or female;
positioning the device of claim 1 at a target intraosseous and/or periosteal site in the subject;
wherein the subject is a male and wherein the sex hormone composition comprises a ratio of androgen to estrogen of at least 10:1 and optionally up to 20:1, or
wherein the subject is a female and wherein the sex hormone composition comprises a ratio of androgen to estrogen of at least 1:5 and optionally up to 1:3;
the sex hormone composition eluting from the implantable structure for a period of time; and
the sex hormone composition promoting bone remodeling at the target site.

22. The method of claim 21, wherein levels of the estrogen and the androgen are at least 5 times higher at an anatomical region associated with the target site than at anatomical regions remote from the target site during the period of time.

23. (canceled)

24. (canceled)

25. The device of claim 17, wherein the device omits other growth factors.
